(19) United States
(12) Patent Application Publication
(10) Pub. No.: US 2021/0161553 A1
(43) Pub. Date: Jun. 3, 2021

(54) SURGICAL INSTRUMENT STABILIZATION DEVICE

(71) Applicant: HOWARD UNIVERSITY, Wahington, DC (US)

(72) Inventors: Roger C. LIN, Wahington, DC (US); Olmide A. OLULADE, Wahington, DC (US); Shannon A. SULLIVAN, Wahington, DC (US); Hamza HAIDER, Wahington, DC (US); Alexander W. SONG, Wahington, DC (US)

(73) Assignee: HOWARD UNIVERSITY, Wahington, DC (US)

(21) Appl. No.: 17/046,449

(22) PCT Filed: Apr. 12, 2019
(86) PCT No.: PCT/US2019/027294

§ 371 Date: Oct. 9, 2020

Related U.S. Application Data
(60) Provisional application No. 62/656,736, filed on Apr. 12, 2018.

(51) Int. Cl. A61B 17/34

(52) U.S. Cl. CPC A61B 17/3403; A61B 2017/00951

(57) ABSTRACT

In some embodiments, apparatuses and methods are provided herein useful to guide and support a medical device, such as a needle, relative to the skin of a patient. In some embodiments, the apparatus is attached to the skin of a patient through suction or adhesive materials, and allows for accurate placement of the medical instrument.

### RELATED APPLICATIONS

The present application is a non-provisional application of U.S. Application 62/656,736, filed Apr. 12, 2018, which is hereby incorporated by reference as if fully set forth herein.

### FIELD

This invention relates generally to medical devices, and more specifically to methods and apparatus for stabilizing or bracing instruments for controlled contact with a target area of a patient.

### BACKGROUND

Many surgical and other medical procedures require instruments to be accurately targeted to a particular site on the patient and held in place while a procedure is performed. For instance, CT or MRI-guided biopsy is a commonly performed procedure by interventional radiologists to remove tissue samples for further examination. The procedure involves inserting a biopsy needle into the patient and using CT or MRI images to ensure accurate placement of the needle before removing the tissue sample. These biopsy procedures can typically last from fifteen minutes to a couple hours, depending on the complexity of the procedure. One recurring problem with these procedures is displacement of the biopsy needle and difficulty in estimating the precise angle at which the biopsy needle is placed. Not having a secure foundation for the biopsy needle is especially problematic for procedures that require insertion of the biopsy needle at an angle that is more horizontal, as the weight of the needle can displace the tip. These problems lead to prolongation of the procedure due to readjusting and repositioning of the biopsy needle. They can also cause the patients additional pain during the procedure. The present invention offers a solution to the ‘problem of biopsy needle stabilization and obtaining a precise angle of entry.

Several patents existing for biopsy needle stabilization devices. In U.S. Pat. No. 8,308,740 B2 to Tolley et al., a frusto-conical frame structure is provided with an adhesive base that stabilizes the biopsy needle while inserted in the patient. The top of the frame contains a planar flexible diaphragm that is spaced apart from the base. The diaphragm is punctured by the needle and releasably grips and supports the shaft of the needle once punctured. The diaphragm allows pivoting of the needle, and supports the needle once inserted into the patient. However, this device does not assist in accurate and precise placement of the biopsy needle or measurement of the angle of insertion.

In US 2009/0247859 A1 to Daum et al., a rigid but adjustable arm is mounted to an MM table and has the capability to be used for prostate biopsies while a patient lies in the prone position. The apparatus is fixed to one portion of the MRI table and has several movable components that allow for three-dimensional positioning for desired needle entry into the patient's anus. However, while this device provides needle stabilization and a secure foundation of the apparatus to the MM table, it has many components, is relatively complicated to operate and requires adjustment at several points, is fixed in place on a large table, and is relatively expensive to manufacture.

US2007/0149878A1 to Hankins et al. provides a manner of securing a needle position and finding an accurate and precise angle for a medical instrument, such as a biopsy needle, relative to the patient's position. It utilizes multiple components and mechanisms including a positioning arm and a protractor. However, it includes several components, is relatively complicated to operate and requires adjustment at several points, and is relatively expensive to manufacture.

A need remains for improved stabilization devices.

### SUMMARY

Generally speaking, apparatuses and methods are provided herein useful for stabilizing and/or positioning instruments such as needles during medical procedures, especially procedures requiring penetration of an instrument through the skin of a patient. The apparatus may be positioned on the skin of a patient in order to receive one or more instruments, such as one or more needles or needle guides. The needles used with the apparatus may be solid or hollow. In some forms, the apparatus assists in positioning the instrument and guiding it to a target site. In some forms, the apparatus is attached to the surface of a patient's skin without the need to be mounted or connected to a table or other surface, such as by suction and/or adhesion directly to the patient's skin. The apparatus may be rigid, semi-solid, or a combination thereof.

In some embodiments, the apparatus serves to position and support a needle during and after insertion into a patient. In some forms, the apparatus may include features, such as markings or pre-positioned structures, to act as guides for positioning an instrument and/or allow the user to advance an instrument toward a patient along a predetermined path. In some forms, the apparatus includes features such as markings to align an advancing instrument with a target site and/or select a specific entry angle for the instrument.

### BRIEF DESCRIPTION OF THE DRAWINGS

Disclosed herein are embodiments of systems, apparatuses and methods pertaining to aligning instruments for interaction with a patient. This description includes drawings, wherein:

FIG. 1 is one example of an apparatus for positioning, inserting, and supporting a needle in accordance with some embodiments.

FIG. 2 is another apparatus in accordance with several embodiments, secured in place primarily by adhesive compounds.

FIG. 3 is an elastomeric apparatus in accordance with some embodiments.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to various embodiments, systems, apparatuses and methods are provided herein useful for stabilizing and/or positioning instruments such as needles during medical procedures, especially procedures requiring penetration of an instrument through the skin of a patient. In some forms, an apparatus serves as a support device for stabilizing a medical instrument with respect to the skin of a human or animal. In some forms, the apparatus has a rigid or semi-rigid structure that attaches to the skin of a human or animal patient via suction, adhesion, and/or anchoring with devices such as straps or surgical tape. The apparatus attaches to the surface of human or animal skin without the need to be mounted or connected to a table or other surface. In some embodiments, suction or adhesion alone maintains the apparatus in place on the skin of a patient. In some embodiments, a combination of suction and adhesion keeps the apparatus in place and in contact with the skin of a patient.

In some embodiments, the apparatus serves to position and support a needle during and after insertion into a patient. In some forms, stabilization of needle position is accomplished by reducing uncontrolled movement of the needle, supporting the weight of a needle, and accommodating multiple angles of needle entry while minimizing repositioning the apparatus. In some embodiments the apparatus may be lightweight, and may be operated by a single user.

In some embodiments, the stabilization apparatus is formed from low-cost materials such as plastic and/or elastomeric materials. Suitable rigid and semi-rigid plastic materials include acrylic polymers and copolymers, nylon, and the like. In some forms, the apparatus may comprise 3-D printed material, such as 3-D printed nylon. Suitable elastomeric materials include, but are not limited to, silicone and silicone-containing materials.

In some forms, the apparatus has a generally circular base and forms a dome shape through which the needle will pass. The apparatus may also be a cylinder, cube, pyramid, polyhedron, frustum, or other shape, or may even have an irregular or asymmetric form. In some forms, the apparatus comprises a rigid outer shell with perforations, and may contain a hollow space within the device. In some forms, the perforations of the rigid outer shell may be filled with a semisolid or elastomeric material. In other embodiments, the apparatus may comprise a semisolid block of viscous material, such as an elastomer. In some embodiments, a device may comprise a combination of rigid outer shell and inner elastomeric material.

In some embodiments, the apparatus includes one or more access windows or points of insertion to assist in guiding and positioning a needle. In some forms, a plurality of access points are provided to permit selection of one of a plurality of pre-determined entry angles to assist in accurate and precise needle insertion. For instance, a difficult to penetrate shell may include a plurality of bores or other openings through which an instrument may be easily inserted. Alternatively, in some forms the apparatus may include predetermined points which give way as an instrument is inserted. In some alternative forms, the entire apparatus is easily penetrated and allows insertion at any point from any angle.

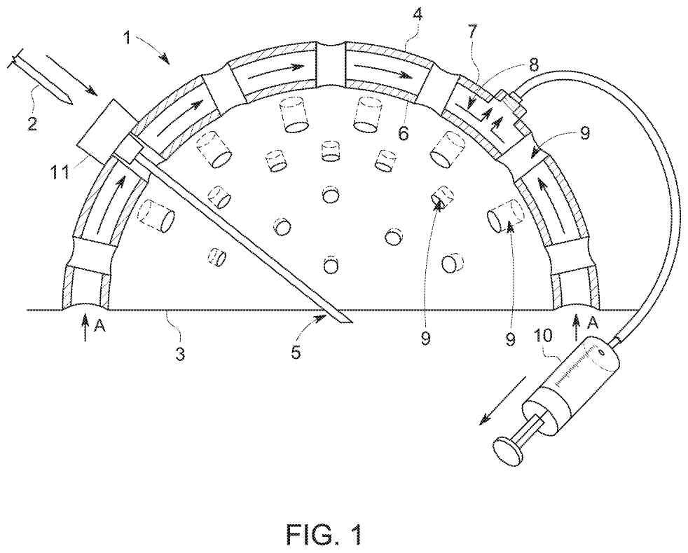

FIG. 1 illustrates one embodiment of a stabilization apparatus 1 that supports and guides a needle 2 to a specific site on a patient's skin 3. In the illustrated form, the needle holder is in the form of a dome 4 that will be placed on top of the needle entry site 5. The dome may be made in part or in whole of rigid plastic. The dome may consist of two layers, with an inner shell 6 and an outer shell 7 forming a space 8 therebetween. The inner and outer shells may be connected by multiple cylindrical passageways 9 that will allow entry of the needle 2, and optionally a needle guide 11. In some embodiments, only a single opening or passageway is provided. The space 8 between the two layers may be sealed except at the lower end that contacts the skin, and the lower rim of the shell may be formed as a continuous structure in order to allow suction to adjacent skin. Air within the space 8 between inner and outer shells may be removed by a suction device, such as via the illustrated syringe 10 placed in communication with the space between the inner and outer shell. Sliding the piston of the syringe 10 backward draws in air from the domes inner space 8 as shown by the series of arrows in the space 8 between shell layers to create a seal with the patient's skin via suction as illustrated by arrows A. The space between the shells may be open or partially open at the bottom in order to facilitate adhesion to the patient's skin. By evacuating air from the space 8 between the shells, a seal is formed with the patient's skin in a ring around the target site, which does not cause substantial deflection of the patient's skin at the target site. The seal formed between the shells and the patient's skin provides stability to the design and allows the dome to remain in place on the patient's skin under the weight of the needle, so that the apparatus supports and hold the needle in place. A needle guide may also be associated with the dome and positionable within one or more of the plurality of passageways. In some embodiments, a plurality of needle guides may be provided with the dome. In some embodiments, one or more needle guides are integral with the dome structure to provide a predetermined access point for the needle. In some embodiments the needle guide may pierce the patient's skin, while in other embodiments it may extend only partially to the target site.

In alternative embodiments the air within the dome of the inner shell may be evacuated if the passageways 9 are filled with elastomeric material to form a seal between the inner surface of the dome and the patient's skin. In such an embodiment, only a single shell need be provided.

In the form illustrated in FIG. 1, the multiple passageways provide the physician with various angles for optimal needle placement. The passageways may form an array of regularly spaced openings to provide specified entry angles, which may be marked on the outer surface with symbols, shapes, lines, writing, or the like in order to enable a user to select a specified angle and accurately position the needle at the specified angle. In some embodiments, the dome structure is transparent or translucent, allowing visualization of the needle as it passes through the structure. Markings may be formed by ink, etching, embossing, or in any other manner. In some embodiments, the passageways and/or interior space of the dome may be filled with a gel or elastomeric material.

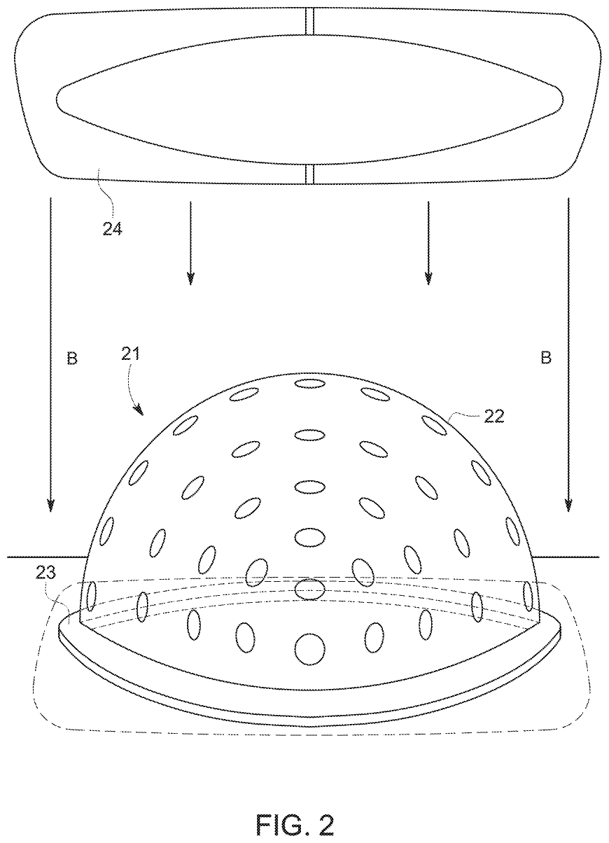

In FIG. 2, another embodiment is shown in which the stabilization apparatus 21 includes a dome structure 22 having a plurality of passageways and a ridge 23 or lip at its base. The dome may be placed on top of a needle entry site and fastened to the patient's skin using adhesive materials. The base of the dome may be coated on its underside with an adhesive composition that adheres to the patient's skin. In addition, or alternatively, adhesive tape 24 may be applied to at least a portion of the upper surface of the lip so that it extends across the patient's skin, anchoring the dome in place. In some embodiments, tape is lowered over the apparatus in the direction of Arrows B and positioned across substantially the entire upper surface of the lip of the device. In some embodiments, the tape placed over the device may include a window sized and configured to receive the dome portion of the stabilization apparatus and contact the upper surface of the lip portion, so that only a single piece of tape is required to secure the apparatus to the patient. Suitable tapes for such applications include Tegaderm™ products sold by 3M Corp. A lip 23 of the types shown in FIG. 2 may also be added to an apparatus of the type shown in FIG. 1 to provide multiple methods for affixing the apparatus.

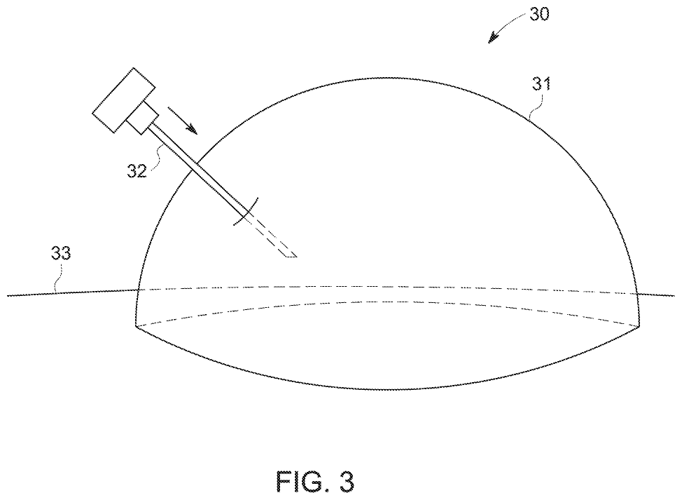

In FIG. 3, an embodiment is shown in which the stabilization apparatus 30 comprises an elastomeric or gel material. The material forms a viscous mass of material capable of supporting an instrument. In the illustrated form, the material is shaped in the form of a dome 31, although other shapes are also possible. The dome is manufactured from a semi-solid material such as silicone, and is preferably dense and strong enough to hold a needle guide and/or needle in position along an insertion path. In some forms, the material also provides friction with the patient's skin for added stability. The weight of the dome allows it to sit in place on the patient with increased traction. The user may insert, remove, and reinsert a needle and optionally a needle guide 32 in various positions to obtain a desired angle. In some forms, a needle guide may be pre-positioned within the apparatus while in other embodiments a needle guide may be inserted by a user at desired locations and angles, or can be entirely omitted. In some embodiments, the semisolid material is transparent or translucent to allow visualization of the needle as it passes through the structure. The external surface of the structure may be provided with markings or indentations, such as lines, shapes, letters, numbers, or symbols, to indicate particular angles or positions. Markings or indentations on the apparatus, such as on the upper or lower surface, may also assist in positioning the center of the device over the target site on the patient and/or guiding the instrument to the target site. Markings may appear on the dome and/or bae of the apparatus, and may be formed by the application of inks or other materials, etching, embossing, or other methods.

In some embodiments, the apparatuses described herein are useful in methods of fixing a needle in place relative to a patient. In some forms, the methods involve attaching a shell structure to a patient's skin via suction and/or adhesion, inserting an instrument through a perforation of the shell, and advancing the instrument into and below the patient's skin. In some forms, the method further includes inserting a guide into the structure prior to insertion of the instrument, the guide forming a passage wide enough for receiving the instrument therein. In some embodiments, the needle may be positioned in part within the shell prior to attachment of the shell to the patient. In some forms, such methods include attaching a mass or block of material to a patient's skin via adhesion, inserting an instrument, such as a needle, through the material, and advancing the instrument toward (and in some cases into and/or below) the patient's skin. In some forms, methods involve placing an apparatus against a patient's skin without attachment and aligning an instrument with the apparatus in order to guide the instrument to the patient's skin.

Those skilled in the art will recognize that a wide variety of other modifications, alterations, and combinations can also be made with respect to the above described embodiments without departing from the scope of the invention, and that such modifications, alterations, and combinations are to be viewed as being within the ambit of the inventive concept.

What is claimed is:

1. A device for stabilizing a medical instrument relative the skin of a patient, the device comprising:
at least one shell;
at least one passageway through the shell; and
a continuous lower rim of the shell.

2. The device of claim 1, further comprising a second shell, with a hollow space between the at least one shell and the second shell.

3. The device of claim 1, wherein the shell comprises a hollow space.

4. The device of claim 1, wherein the shell is generally dome-shaped.

5. The device of claim 1, wherein the shell is made of a rigid plastic.

6. The device of claim 1, wherein the shell is made of an acrylic polymer.

7. The device of claim 1, further comprising a suction device for removing air from a portion of the device.

8. The device of claim 1, further comprising an adhesive material for fixing the device to the skin of the patient.

9. The device of claim 1, further comprising an elastomeric material.

10. A device for stabilizing a medical instrument relative the skin of a patient, the device comprising:
a viscous mass of material having a lower surface configured for contact with skin of a patient;
at least one marking on an upper surface of the viscous mass of material for positing a medical instrument; and
an adhesive material to affix the viscous mass of material to the skin of the patient.

11. A method of positioning a medical instrument, the method comprising:
positioning a support device against skin of a patient;
evacuating air from at least a portion of the support device; and
disposing the medical instrument through the support device.

12. The method of claim 11, wherein the medical instrument is a needle and the needle penetrates the skin of the patient after being disposed through the support device.

13. The method of claim 11, wherein the support device comprises a rigid shell.

14. The method of claim 11, further comprising attaching the support device to the skin of the patient using an adhesive material

15. The method of claim 14, wherein the support device comprises a rigid shell.

16. The method of claim 14, wherein the support device comprises a viscoelastic mass.

17. The method of claim 16, wherein the support device is generally dome-shaped.
